(19) United States
(12) Patent Application Publication
(10) Pub. No.: US 2026/0083312 A1
(43) Pub. Date: Mar. 26, 2026

(54) AN IMPROVED ENDOSCOPE TIP

(71) Applicant: BIORAD MEDISYS PRIVATE LIMITED, Pune (IN)

(72) Inventors: Debasish PRADHAN, Pune (IN); Anshuman MATHUR, Pune (IN); Aman SAXENA, Pune (IN)

(21) Appl. No.: 19/113,922

(22) PCT Filed: Sep. 22, 2023
(86) PCT No.: PCT/IN2023/050881

§ 371 Date: Mar. 21, 2025

(30) Foreign Application Priority Data
Sep. 22, 2022 (IN) 202221054413

(51) Int. Cl. A61B 1/307; A61B 1/00; A61B 1/05; A61B 1/06; A61B 18/00; A61B 18/26

(52) U.S. Cl. CPC A61B 1/307; A61B 1/00147; A61B 1/05; A61B 1/0684; A61B 18/26; A61B 2018/00517; A61B 2018/00642; A61B 2018/00773; A61B 2018/00791; A61B 2018/00898; A61B 2018/00982; A61B 2560/0223

(57) ABSTRACT

In one embodiment, a ureteroscope for ureteroscopic laser lithotripsy is disclosed. The ureteroscope comprises a handle, a disposable section made of a flexible cannula constructed of polyamide intertwined with 316L stainless steel wires with a specific diameter of 2.5 mm, a trapezoidal shaped camera, a distal tip and an LED module arrangement composed of four LEDs, each precisely situated at a 90-degree interval around the camera, and a temperature sensor situated at the distal tip, calibrated to detect a temperature range from 25° C. to 50° C., a pressure sensor located at the distal tip with a sensitivity range of 0 to 40 mmHg for regulating intrarenal pressure and a Video Processing Unit (VPU) with a processing speed of 2.5 GHz, configured to receive and process data from the sensors and the camera.

### CROSS-REFERENCE TO RELATED APPLICATION

This application claims the benefit of Indian Provisional Application No. 202221054413 filed Sep. 22, 2022, which is incorporated herein by reference in its entirety.

### FIELD OF THE SUBJECT MATTER

The present subject matter relates generally to the field of medical instruments. More specifically, the present subject matter relates to an endoscopic device particularly a ureteroscope having a tip structure with a distal camera LED module and sensors.

### BACKGROUND

Endoscopic devices for surgical or diagnostic procedures are already known. Endoscopes have gained widespread acceptance in the medical community because they provide a means for performing surgery with minimal trauma to the patient while allowing the physician to view the internal anatomy of the patient. Over the years, many endoscopes have been developed and classified according to specific applications, such as ureteroscopes, cystoscopes, colonoscopes, laparoscopes, upper gastrointestinal endoscopes, and the like. The endoscope may be inserted into a natural opening in the body, or through an incision in the skin. A conventional endoscope is generally an instrument with a handle and an insertion part (or a reusable or a disposable part in case of split endoscopes connected to each other by some means) wherein, various working components for observation like an optical or electronic imaging system and the like are provided at a distal end of the insertion part, a proximal end at handle with controls for manipulating the tool and devices for viewing the image is provided, and a solid or tubular elongate shaft connecting the ends.

To use an endoscope, a physician inserts the distal end into a patient through a natural orifice or an artificial incision to diagnose or treat a disease state of the patient using the working components at a distal end of the insertion part. The proximal end remains outside the patient and typically connects to an eyepiece, video monitor, or other equipment. The insertion part uses a complex lens system for transmitting the image from the distal tip of the endoscope to a viewer. The lens system is typically a relay lens system in the case of rigid endoscope or a bundle of fiber optics or an objective lens system in the case of flexible endoscopes. In the case of both rigid and flexible conventional endoscopes, the lens or fiber optic system is expensive. In more currently developed endoscopes, discrete illuminators such as light-emitting diodes (LEDs) have been incorporated for providing illumination.

Kidney stones (known as ureteral calculi in medical terminology) are a common medical problem that negatively impacts millions of individuals worldwide. Laser Lithotripsy procedures are getting popularity in today's Urology world primarily because of their ability to break the stones into tiny fragments and dusts sizing less than 0.2 mm. In addition, laser lithotripsy operation need to be performed in a water-containing environment (such as saline, contrast agents, etc.) the use of water in the operation can cause the change of the internal pressure of the organ, and can cause great harm to the human body when the internal pressure of the organ is too high or too low. High intraluminal pressure, in some instances, may make it easier for bacteria and endotoxins to be absorbed into the blood, resulting in postoperative fever. High pressure may also cause lymph node and venous reflux which may lead to fluid leakage, post-operative pain, urosepsis and renal injury. Further, rapidly rising intrarenal pressures may increase the risk of extravasation with the potential to cause pyelosinus, pyelovenous, and/or pyelolymphatic backflow of an irrigant provided to the kidney which severely endangers the life of the patient.

Furthermore, in Lithotripsy Procedures, if there is insufficient irrigation, the temperature within the body cavity may rise to unsafe levels. In addition, the lens module and the LED generate heat inside the tip body. At present, most of the tip parts of the insertion parts of the existing medical endoscopes are not provided with temperature measuring devices, and the temperature of the tip parts and the temperature inside a cavity cannot be monitored after the endoscope is operated to enter the cavity. Tip heating not only causes discomfort to the patient but may also affect the performance of some of the electronic components inside the tip. Failure of a component to operate due to too high a temperature is also known. In particular, under high temperature conditions, LEDs exhibit reduced brightness and a shift in chromaticity towards blue. In general, imagers experience higher noise and a change in image characteristics such as hue, saturation, brightness, and contrast at higher temperatures.

While the state-of-the-art relating to endoscopes is relatively advanced today, there are still recurring problems and to overcome the above problems, there is a need for monitoring of the pressure and temperature in clinical surgery, which is directly related to the surgical safety of patients. This subject matter is therefore directed towards providing an improved endoscope which is relatively simple in design and structure and is highly effective for its intended purpose.

### SUMMARY

Embodiment of the present disclosure present technological improvements as solutions to one or more of the above-mentioned technical problems recognized by the inventor(s) in conventional systems.

In one embodiment, a ureteroscope for ureteroscopic laser lithotripsy is disclosed. The ureteroscope comprises a handle portion designed for ergonomic single-handed operation, a disposable section made of a flexible cannula constructed of polyamide intertwined with 316L stainless steel wires with a specific diameter of 2.5 mm, a trapezoidal shaped camera specifically tailored for a 7.5 Fr endoscope with a resolution of 1920×1080 pixels, a distal tip and an LED module arrangement composed of four LEDs, each precisely situated at a 90-degree interval around the camera, having a combined output of 600 lumens to eliminate the requirement for a fibre optics cable and a temperature sensor situated at the distal tip, calibrated to detect a temperature range from 25° C. to 50° C. to monitor cellular damage risk during ureteroscopic laser lithotripsy, a pressure sensor located at the distal tip with a sensitivity range of 0 to 40 mmHg for regulating intrarenal pressure and a Video Processing Unit (VPU) with a processing speed of 2.5 GHz, configured to receive and process data from the sensors and the camera, and to display said data on a monitor with a minimum latency of 10 milliseconds.

In one embodiment, the LED module arrangement and the camera operate synergistically to provide illumination and real-time visualization without reliance on an external light source and the temperature sensor alerts the operator in real-time when detected temperatures approach the upper limit of its calibrated range during ureteroscopic laser lithotripsy procedures. Further, the pressure sensor provides an immediate feedback mechanism for the surgeon by alerting of any abnormal intrarenal pressure conditions. Furthermore, a data transfer system consisting of dual PCBs to ensure rapid communication between the sensors and the VPU. The VPU utilizes a proprietary algorithm to process and enhance raw imagery from the camera, ensuring clarity and sharpness of the displayed visual data. Additionally, the disposable section features a built-in mechanism for quick detachment and attachment, facilitating efficient replacement between procedures.

It will be appreciated by those skilled in the art that features of the present disclosure are susceptible to being combined in various combinations without departing from the scope of the inventive concept thereof as defined by the below mentioned detailed description and drawings. It is understood therefore, that this subject matter is not limited to the embodiments disclosed, but rather modifications are also covered within the scope of the present subject matter as defined by the appended claims.

### BRIEF DESCRIPTION OF THE FIGURES

The summary above, as well as the following detailed description of illustrative embodiment, is better understood when read in conjunction with the appended drawings. For the purpose of illustrating the present disclosure, example constructions of the disclosure are shown in the drawings. However, the present disclosure is not limited to specific methods and instrumentalities disclosed herein. Moreover, those in art will understand that the drawings are not to scale. Wherever possible, like elements have been indicated by identical numbers.

Embodiment of the present disclosure will now be described, by way of example only, with reference to the following diagrams wherein:

FIG. 1 illustrates a perspective view of a two-piece ureteroscope device in accordance with an exemplary embodiment of the present disclosure;

FIGS. 2(a) and 2(b) illustrates the perspective view and side views respectively of the distal tip of the two-piece ureteroscope device in accordance with an exemplary embodiment of the present disclosure;

FIG. 3(a)-3(d) illustrates sectional view of the two-piece ureteroscope device showing the transfer of signals with an exemplary embodiment of the present disclosure;

In the above accompanying drawings, a number relates to an item identified by a line linking the number to the item. When a number is accompanied by an associated arrow, the number is used to identify a general item at which the arrow is pointing.

Further the figures depict various embodiments of the present subject matter for purposes of illustration only. One skilled in the art will readily recognize from the following discussion that alternative embodiment of the structures and methods illustrated herein may be employed without departing from the principles of the present subject matter described herein.

### DETAILED DESCRIPTION OF EMBODIMENTS

The following detailed description illustrates embodiment of the present disclosure and manners by which they can be implemented. The words “comprising.” “having.” “containing,” and “including.” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. As used herein, a “plurality” refers to two or more, for example, three or more, four or more, five or more, six or more. Each possibility represents a separate embodiment of the subject matter.

For purposes of the present specification and claims, various relational terms like “top” “bottom,” “proximal,” “distal,” “upper,” “lower,” “front,” and “rear” are used to describe the present subject matter when said subject matter is positioned in or viewed from a given orientation. It is to be understood that, by altering the orientation of the subject matter, certain relational terms may need to be adjusted accordingly.

The person skilled in the art will recognize many variations, alternatives, and modifications of the embodiment of the present disclosure. It should be understood that this subject matter is not limited to the particular methodology, protocols, and the like, described herein and as such may vary. The terminology used herein is for the purpose of describing particular embodiment only and is not intended to limit the scope of the present subject matter, which is defined solely by the claims.

The present subject matter proposes an improved ureteroscope with a combination of disposable and reusable components that solves the problem of calculus on the human body caused by high pressure and high temperature in the existing lithotripsy system during surgery.

Another object of the present subject matter is to provide an improved ureteroscope having a tip structure of a ureteroscope device with a camera and LED module for illumination thereby eliminating fibre optics cable.

Further object of the present subject matter is to provide an improved ureteroscope having a tip structure with a temperature sensor to monitor the temperature of the internal environment of the cavity.

It is still another object of the present subject matter to provide an improved ureteroscope having a tip structure with a pressure sensor making it easy for a doctor during treatment to notice a change in internal pressure.

Yet another object of the subject matter is to provide a ureteroscope device distal tip structure which is simple in structure, easy to assemble with camera module and sensors, easy and convenient to operate, saves operation time and high in working efficiency.

In an aspect of the present disclosure, an improved ureteroscope with a combination of disposable and reusable components solving the problem of calculus on the human body caused by high pressure and high temperature in the existing lithotripsy system during surgery is disclosed. The reusable part and the disposable part are easy to assemble and disassemble. Further, the ddistal tip of the endoscope is integrated with smallest trapezoidal shaped camera and led module arrangement, that helps in building a 7.5 Fr endoscope. A temperature and a pressure sensor is also integrated at the distal tip so that the surgeon can keep an eye on the increasing pressure and temperature during ureteroscopic laser lithotripsy and can control the kidney Renel pressure to avoid sepsis post surgery and can control the rising temperature during ureteroscopic laser lithotripsy to avoid cellular damage. The data is captured and sent to the Video Processing Unit (VPU) via PCB's. The processed data is transferred to monitor that guides the surgeon to control the irritation and control the IRP and maintains the body temperature during ureteroscopic laser lithotripsy.

Additional aspects, advantages, features and objects of the present disclosure would be made apparent from the drawings and the detailed description of the illustrative embodiment. Further, In the table below nomenclature used is provided.

| Number | Nomenclature |
|---|---|
| 100 | ureteroscope device |
| 102 | Handle |
| 104 | disposable portion |
| 106 | distal end |
| 108 | Proximal end |
| 110 | Cannula |
| 112 | distal end |
| 114 | proximal end |
| 116 | lever |
| 118 | unlock button |
| 120 | working channel |
| 122 | Tip |
| 202 | Pressure Sensor |
| 204 | Temperature Sensor |
| 206 | Camera |
| 208 | led module arrangement |
| 302 | First PCB |
| 304 | Second PCB |
| 306 | monitor |
| 308 | signals |

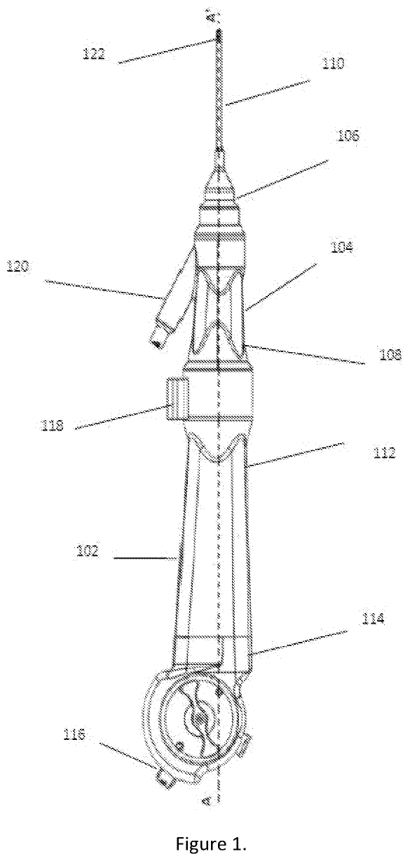

Referring now to the drawings, FIGS. 1-3 a single hand operate two piece endoscope device, in particularly a distal tip of ureteroscope integrated with camera and LED module arrangement along with sensors is illustrated accordingly to present subject matter. It should be noted that FIG. 1-3 are merely examples. A person skilled in the art will recognize many variations, alternatives, and modifications of the embodiment of the present disclosure.

Referring now to the drawings, FIG. 1 illustrates a ureteroscope device 100 in an attached configuration comprising a reusable portion particularly a handle 102 and a disposable portion 104. The disposable portion 104 has a distal end 106 and a proximal end 108, wherein the disposable portion 104 is adapted to be inserted into a location to be observed inside a subject. A cannula 110 made up of polyamide braided with metal wires is disposed from the distal end 106 wherein the cannula can be flexible and may include one or more lumens (not shown) along with a tip 122 at bending portion which can be articulated and deflected in any direction along or about longitudinal axis A-A′. A handle 102 of the endoscope device 100 comprising of a distal end 112 and proximal end 114 is configured to control the movement of cannula 110 in one or more directions relative to longitudinal axis A-A′. The handle 102 is made up of high-performance thermoplastics like polysulfone and ergonomically designed to easily operate using one hand by the user. The user can move the bending portion in one or more directions with the help of a lever 116. In order to detach the handle 102 from the disposable portion 104, an unlock button 118 is provided. Further a working channel 120 is provided on the disposable portion 104.

FIG. 1 is merely an example. A person skilled in the art will recognize many variations, alternatives, and modifications of the embodiment of the present disclosure. The material used in manufacturing of the ureteroscope can be different than the family of high-performance thermoplastics like polysulfone and the like as used in the present subject matter. The material for cannula can be of different material other than polyamide.

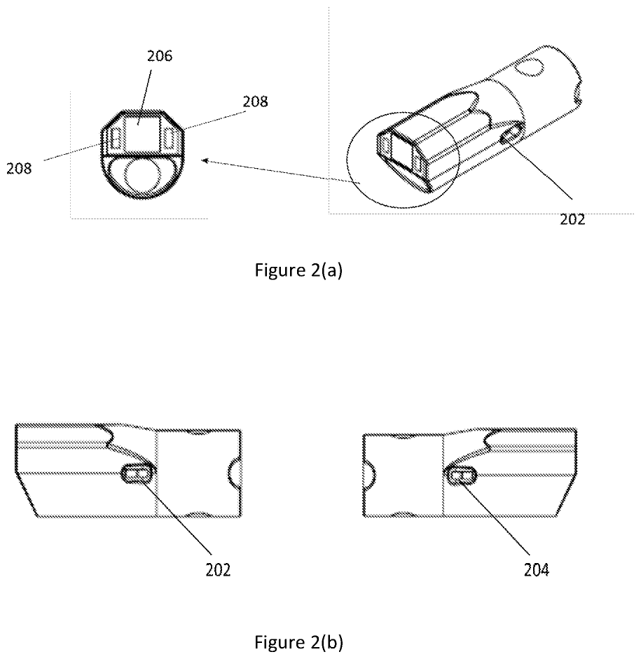

Now referring to FIGS. 2(a) and 2(b), tip 122 of the ureteroscope device 100 is illustrated. The Distal tip 122 is integrated with a pressure sensor 202 and a temperature sensor 204 is illustrated. Further, the tip 200 is integrated with a small trapezoidal shaped camera 206 and 208 that helps in building a 7.5 Fr endoscope. The size of the camera 206 is kept small so that it would be easy to insert it in any tiny orifice of the body when integrated with the tip eventually causing lesser pain to the patient and to access the smallest of passage during surgery. Further, a plurality of Light Emitting Diode (LED) 208 on each side of the camera is provided to illuminate the area when the camera captures the information.

A tiny sized absolute pressure sensor 202 is placed at the left side of the distal tip 122 that captures the data and sends it to the VPU (not shown) via a plurality of PCB's (explained later in description) thereby guiding the surgeon to control the irrigation and Intrarenal Pressure (IRP). A tiny sized temperature sensor 204 is also placed at the right side of the distal tip 122 that captures the data and sends it to the VPU (not shown) via a plurality of PCB's (explained later in description) which helps the surgeon to control the irrigation and maintain the body temperature during ureteroscopic laser lithotripsy to avoid cellular damage.

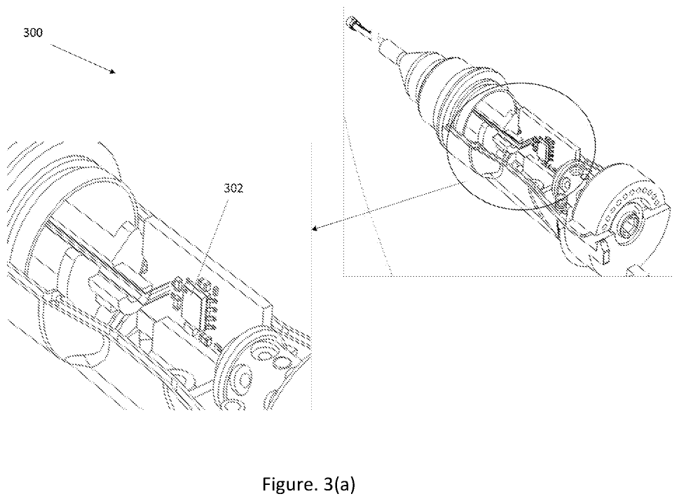

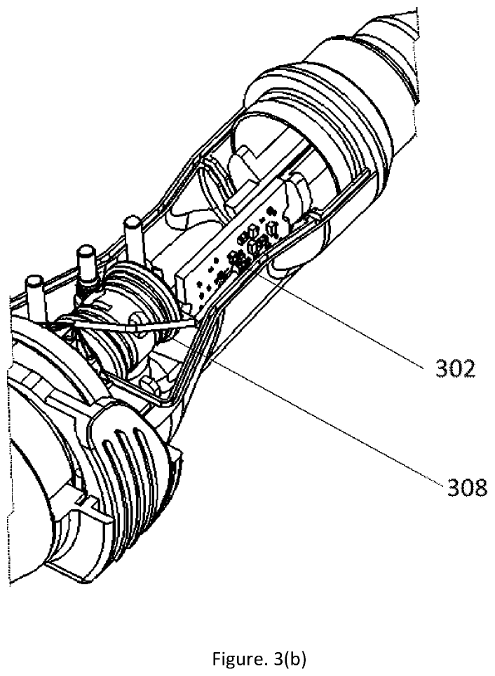

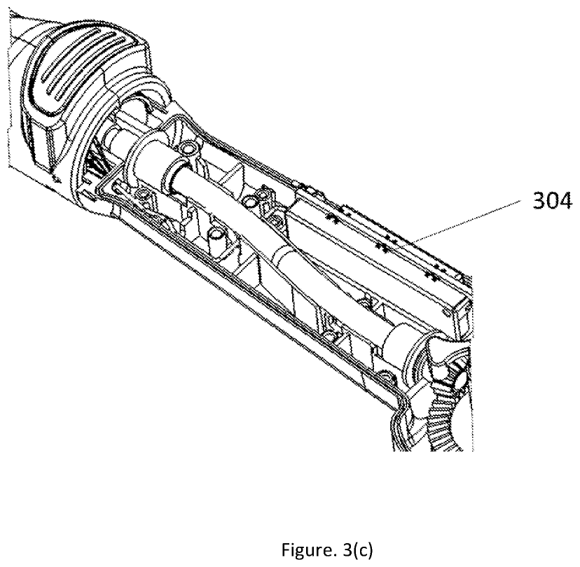

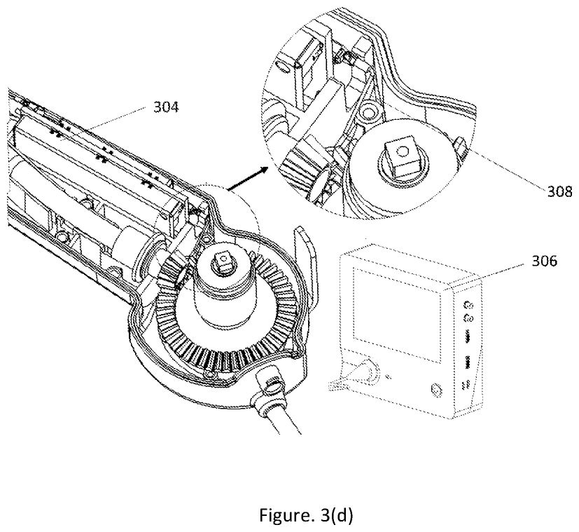

FIG. 3(a)-3(d) illustrates the sectional view of the two piece ureteroscope device showing the transfer of signals. As can be seen in FIG. 3(a), the temperature sensor 204, pressure sensor 202 and the LED's 208 are connected to the first PCB 302 present at the disposable portion. From there the signals 308 are transferred to the second PCB 304 present at the reusable portion of the handle as shown in FIGS. 3(b) and 3(c). The signals 308 are further transferred to the VPU (not shown) via second PCB 304 and the processed data is shown on the monitor 306 as shown in FIG. 3(d).

The camera captures the raw images and pass it to the Video Processing Unit (VPU), where the signals are converted from analogue to digital. This captured information from the camera is processed by the VPU and is transferred to the monitor (not shown) through the output port. The monitor displays the information captured by the camera. The purpose of the White light LED's is to provide the illumination when the camera is capturing the information. It helps to provide the required brightness to capture the information by the camera and come up with a required image resolution.

FIG. 1-3 are merely examples. A person skilled in the art will recognize many variations, alternatives, and modifications of the embodiment of the present disclosure.

It is observed that the ureteroscope device has provided the much needed advantage in surgery which avoids any discomfort to the patient as was the case in conventional methods. Along with these advantages, the compact size of the camera ensures accessing smaller passages.

The present subject matter has both technical as well as economic significance with respect to conventional devices.

While a particular embodiment of the subject matter has been illustrated and described, modifications thereof will readily occur to those skilled in the art. It is understood that the various embodiment, details, and constructions of the ureteroscope device and their features described above and illustrated in the attached figures may be interchanged among the various embodiment while remaining within the scope of the subject matter. Additionally, it is understood that various modifications could be made to any of the ureteroscope device and/or elements described herein above while remaining within the scope of the subject matter.

1. An ureteroscope for ureteroscopic laser lithotripsy, wherein the ureteroscope, comprises:
a handle (102) portion designed for ergonomic single-handed operation;
a disposable portion (104) made of a flexible cannula (110) constructed of polyamide intertwined with 316L stainless steel wires with a specific diameter of 2.5 mm;
a trapezoidal shaped camera (206) specifically tailored for a 7.5 Fr endoscope with a resolution of 1920×1080 pixels;
a distal tip (122);
an LED module arrangement (208) composed of four LEDs, each precisely situated at a 90-degree interval around the camera (206), having a combined output of 600 lumens to eliminate the requirement for a fibre optics cable;
a temperature sensor (204) situated at the distal tip (122), calibrated to detect a temperature range from 25° C. to 50° C. to monitor cellular damage risk during ureteroscopic laser lithotripsy;
a pressure sensor (202) located at the distal tip (122) with a sensitivity range of 0 to 40 mmHg for regulating intrarenal pressure; and
a Video Processing Unit (VPU) with a processing speed of 2.5 GHz, configured to receive and process data from the sensors and the camera, and to display said data on a monitor (306) with a minimum latency of 10 milliseconds.

2. The ureteroscope of claim 1, wherein the LED module arrangement (208) and the camera operate synergistically to provide illumination and real-time visualization without reliance on an external light source.

3. The ureteroscope of claim 1, wherein the temperature sensor (204) alerts the operator in real-time when detected temperatures approach the upper limit of its calibrated range during ureteroscopic laser lithotripsy procedures.

4. The ureteroscope of claim 1, wherein the pressure sensor (202) provides an immediate feedback mechanism for the surgeon by alerting of any abnormal intrarenal pressure conditions.

5. The ureteroscope of claim 1, further incorporating a data transfer system consisting of dual PCBs to ensure rapid communication between the sensors and the VPU.

6. The ureteroscope of claim 1, wherein the VPU utilizes a proprietary algorithm to process and enhance raw imagery from the camera (206), ensuring clarity and sharpness of the displayed visual data.

7. The ureteroscope of claim 1, wherein the disposable portion (104) features a built-in mechanism for quick detachment and attachment, facilitating efficient replacement between procedures.
